(19) United States
(12) Patent Application Publication
(10) Pub. No.: US 2015/0175954 A1
(43) Pub. Date: Jun. 25, 2015

(54) Method for Producing a Nutrient Additive Using a Microalga

(71) Applicant: AJINOMOTO CO., INC., Tokyo (JP)

(72) Inventors: Kazuhiko Kunita, Kanagawa (JP); Tomoaki Tanaka, Kanagawa (JP); Yoko Kuwabara, Kanagawa (JP); Yoshihiro Usuda, Kanagawa (JP)

(73) Assignee: AJINOMOTO CO., INC., Tokyo (JP)

(21) Appl. No.: 14/551,952

(22) Filed: Nov. 24, 2014

Related U.S. Application Data
(63) Continuation of application No. PCT/JP2013/064497, filed on May 24, 2013.
(60) Provisional application No. 61/651,636, filed on May 25, 2012.

(30) Foreign Application Priority Data
May 25, 2012 (JP) 2012-119763
Dec. 11, 2012 (JP) 2012-270508

(51) Int. Cl. C12N 1/12; C12P 7/64; C12P 1/00

(52) U.S. Cl. CPC C12N 1/12; C12P 1/00; C12P 7/6409

(57) ABSTRACT

A medium component for more efficiently culturing a microorganism or microalga is provided. A nutrient additive useful in culturing a microorganism or microalga is produced by culturing a microalga in a medium to produce biomass derived from the microalga in the medium, adding an acid to the biomass to hydrolyze the biomass, and preparing the hydrolysate of the biomass as a nutrient additive.

This application is a Continuation of, and claims priority under 35 U.S.C. §120 to, International Application No. PCT/JP2013/064497, filed May 24, 2013, and claims priority therethrough under 35 U.S.C. §119 to Japanese Patent Application No. 2012-119763, filed May 25, 2012, U.S. Provisional Patent Application No. 61/651,636, filed May 25, 2012, and Japanese Patent Application No. 2012-270508, filed Dec. 11, 2012, the entireties of which are incorporated by reference herein.

### BACKGROUND OF THE INVENTION

1. Field of the Invention

The present invention relates to a method for culturing a microalga. Microalgae are used in various fields, such as the fields of productions of bio-fuels, foods, feed additives, drugs, and physiologically active substances.

2. Brief Description of the Related Art

Microalgae can fix carbon dioxide to produce organic substances, and they are the primary producers of organic substances on the earth. Microalgae have various uses. Historically, Spirulina, which is one kind of microalga, was eaten in South America and Africa for medical purposes, and the nutrients and physiologically active substances present in this microalga are currently being studied. Similarly, Chlorella, Dunaliella, Euglena, and so forth are also used as health foods, mainly in Japan. Bivalves are known to feed on microalgae, and culture methods utilizing microalgae as feed for larvae of oyster, short-necked clam, blue mussel, and sea urchin are desired. For example, a culture method in which spats of oyster are raised using microalgae, such as Chaetoceros, cultured in a closed system tank as feed is known. Furthermore, microalgae have been suggested to be suitable in the breeding of livestock for meat including bovines, as they contain saccharides, proteins, lipids, and minerals in nutritionally good balance, and such uses are currently being explored.

It has become clear that various components produced by microalgae also have physiological activities and useful functions (Cardozo, K. H. et al., 2007, Comparative Biochemistry and Physiology-C Toxicology and Pharmacology, 146:60-78). For example, DHA (docosahexaenoic acid) is found in microalgae, and is known to be an unsaturated, or omega fatty acid that suppresses blood cholesterol level and prevents arteriosclerosis. Therefore, methods have been studied and developed for industrially culturing microalgae to produce DHA in a large scale, and purifying DHA therefrom. Furthermore, microalgae can be various colors, and the green color of microalgae is due to chlorophyll. There are also many species presenting such colors as red, orange, and yellow, and it is known that these colors are due to carotenoids, which are natural pigments. It is becoming clear that these carotenoids exhibit many kinds of physiological activities, such as an anti-oxidation action, and uses thereof for functional foods, cosmetics, and so forth are being studied. For example, it has been reported that astaxanthin, which is abundantly produced by Hematococcus microalgae, has a high anti-oxidation action as a physiological function, and thus can defend living bodies from ultraviolet radiation or hyperoxidation of lipids in blood. Thus, industrial implementation of astaxanthin production based on culture of microalgae is being studied.

Depletion of fossil fuels is a recognized problem in human society. Alternatives to petroleum are being explored as fuel sources, and include raw materials, such as corn. Bioethanol produced from starch derived from grains such as corn is being developed. However, such use of grains conflicts with their use as food, resulting in increased cost of all cereals, not just corn-based ones. Therefore, development of economically feasible biomass that does not conflict with food uses is desired. Microalgae basically require carbon dioxide, minerals, and light for proliferation, and can produce fats and oils, such as those mainly used as a diesel oil alternative, without any organic carbon source. Moreover, since microalgae culture does not require either fertile land or a cultivation field, nor is it substantially affected by the change of seasons, bio-fuels should be able to be more efficiently produced from microalgae as compared with other biomasses used for producing bio-fuels (Chisti, Y., 2007, Biotechnology Advances, 25:294-306).

However, since microalgae must be produced in a large scale at a low cost especially for the bio-fuel production, large scale production facilities that keep the cost low as compared with other production methods are necessary. Therefore, a low cost method for culturing microalgae is an important and active field of study (Brennan, L. and Owende, P., 2010, Renewable and Sustainable Energy Reviews, 14:557-577).

An active area of research is the efficient use of alga bodies obtained by culture, since when culturing alga bodies, they are not wholly used, which is not the case for foods or feeds. The production cost of alga bodies could be reduced if, after extraction of physiologically active substances, bio-fuels, and the like, the alga body biomass could be re-used for culture of microalgae.

Methods are known for wholly or partially hydrolyzing alga bodies of microalgae with an acid such as sulfuric acid, acetic acid, and lactic acid, or with an alkali, and using the resulting product as a carbon source or a nitrogen source for culture of microorganisms such as bacteria and yeasts (International Patent Publication WO2009/093703, Chinese Patent Laid-open No. 102229895, Japanese Patent Laid-open (Kokai) No. 2011-229439, and Nguyen, Minh Thu, Seung Phill Choi, Jinwon Lee, Jae Hwa Lee, and Sang Jun Sim, 2009, Journal of Microbiology and Biotechnology, 19:161-166). A method of using an alkali decomposition product of a culture residue of yeast or Klebsiella bacteria as a carbon source for mixotrophic culture of Chlorella has also been reported (Chinese Patent Laid-open No. 102311921). However, methods of using acid-hydrolyzed alga bodies as a nutrient in culture of a microalga has not been reported to date.

### SUMMARY OF THE INVENTION

One aspect of the presently described subject matter is to provide a medium component that is effective for more efficiently culturing a microorganism or microalga. Another aspect is to provide a less expensive method for culturing a microorganism or microalga.

The presently described subject matter describes that if biomass derived from a microalga obtained after culture of the microalga, such as alga bodies of a microalga, disruption product of alga bodies, or an alga body residue that remains after extraction of effective ingredients such as bio-fuels therefrom, was subjected to a hydrolysis treatment with an acid, the obtained hydrolysate added to a medium, and the microalga cultured again in the medium, the microalga could be efficiently cultured.

It is an aspect of the disclosed subject matter to provide a method for producing a nutrient additive useful in culturing a microorganism or a microalga, comprising:

a) culturing a microalga in a medium to produce biomass;

b) hydrolyzing the biomass with an acid, producing an hydrolysate; and

c) preparing a nutrient additive comprising the hydrolysate,

wherein the acid is selected from the group consisting of sulfuric acid, hydrochloric acid, nitric acid, phosphoric acid, and combinations thereof.

It is a further aspect of the disclosed subject matter to provide a method as described above, wherein the hydrolysate promotes growth of a microorganism or microalga.

It is a further aspect of the disclosed subject matter to provide a method as described above, wherein the acid is sulfuric acid, and the acid is added in such an amount that the molar ratio of sulfate ions to nitrogen contained in the biomass (SO₄/N) is 0.1 to 10.

It is a further aspect of the disclosed subject matter to provide a method as described above, wherein the acid is hydrochloric acid, and the acid is added in such an amount that the molar ratio of chloride ions to nitrogen contained in the biomass (Cl/N) is 0.1 to 20.

It is a further aspect of the disclosed subject matter to provide a method as described above, wherein the acid is phosphoric acid, and the acid is added in such an amount that the molar ratio of phosphate ions to nitrogen contained in the biomass (PO₄/N) is 0.1 to 100.

It is a further aspect of the disclosed subject matter to provide a method as described above, wherein the acid is nitric acid, and the acid is added in such an amount that the molar ratio of nitrate ions to nitrogen contained in the biomass (NO₃/N) is 0.1 to 100.

It is a further aspect of the disclosed subject matter to provide a method as described above, wherein the acid is sulfuric acid, and the acid is added in such an amount that the molar ratio of sulfate ions to nitrogen contained in the biomass (SO₄/N) is 0.8 to 3.

It is a further aspect of the disclosed subject matter to provide a method as described above, wherein said hydrolyzing is performed at 75 to 130° C. for 5 to 50 hours.

It is a further aspect of the disclosed subject matter to provide a method as described above, wherein said hydrolyzing is performed at 110 to 120° C. for 10 to 32 hours.

It is a further aspect of the disclosed subject matter to provide a method as described above, comprising treating the biomass at 80 to 110° C. for 30 minutes to 2 hours before said hydrolyzing.

It is a further aspect of the disclosed subject matter to provide a method as described above, comprising treating the biomass at 90 to 105° C. for 40 to 90 minutes before said hydrolyzing.

It is a further aspect of the disclosed subject matter to provide a method for producing objective substance(s), comprising:

culturing a microalga or a microorganism in a medium comprising the nutrient additive produced by the method as described above.

It is a further aspect of the disclosed subject matter to provide a method as described above, wherein the objective substance(s) is L-amino acid(s).

It is a further aspect of the disclosed subject matter to provide a method as described above, wherein the objective substance(s) is starch.

It is a further aspect of the disclosed subject matter to provide a method as described above, wherein the objective substance(s) is lipid(s) or fatty acid(s).

### DETAILED DESCRIPTION OF THE EMBODIMENTS

<1> Method for Producing a Nutrient Additive

A method for producing a nutrient additive for culture of a microorganism or a microalga is provided having, at least, the following steps: a) culturing a microalga in a medium to produce biomass derived from the microalga, b) hydrolyzing the biomass by adding an acid to the biomass, and c) preparing a nutrient additive containing, at least, the hydrolysate of the biomass (henceforth also referred to as the “method for producing a nutrient additive”). The nutrient additive produced by that method is also referred to as the “nutrient additive”.

That is, in the method for producing a nutrient additive as disclosed herein, a microalga is cultured in a medium to produce biomass derived from the microalga.

<1-1> Microalga and Culture Thereof

“Algae” can refer to all organisms that perform photosynthesis that generates oxygen, except for Bryophyta, Pteridophyta, and Spermatophyta, which live mainly on the ground. Algae can include various unicellular organisms and multicellular organisms, and specifically can include, for example, organisms classified into cyanobacteria (blue-green algae), which are prokaryotes, and organisms classified into the phylum Glaucophyta, Rhodophyta (red algae), Chlorophyta, Cryptophyta (crypt algae), Haptophyta (haptophytes), Heterokontophyta, Dinophyta (dinoflagellates), Euglenophyta, or Chlorarachniophyta, which are eukaryotes.

“Microalgae” can refer to algae having a microscopic structure, which do not include multicellular marine algae (Biodiversity Series (3) Diversity and Pedigree of Algae, edited by Mitsuo Chihara, Shokabo Publishing Co., Ltd. (1999)). Microalgae can also include algae the cells of which form a colony.

The microalga can be any organism classified into the microalgae as mentioned above.

It is known that some microalgae accumulate fats and oils as storage substances (Chisti Y., 2007, Biotechnol. Adv., 25:294-306), and include algae belonging to the phylum Chlorophyta or Heterokontophyta.

Examples of the algae belonging to the phylum Chlorophyta include those belonging to the class Chlorophyceae, Trebouxiophyceae, Prasinophyceae, Ulvophyceae, Charophyceae, or the like. Examples of algae belonging to Chlorophyceae include, for example, Neochloris algae such as Neochloris oleoabundans (Tornabene, T. G. et al., 1983, Enzyme and Microb. Technol., 5:435-440), Nannochloris algae such as Nannochloris sp. (Takagi, M. et al., 2000, Appl. Microbiol. Biotechnol., 54:112-117), Chlamydomonas algae such as Chlamydomonas reinhardtii, Scenedesmus algae, and Desmodesmus algae. Examples of algae belonging to the class Trebouxiophyceae include, for example, Chlorella algae such as Chlorella kessleri.

Examples of algae belonging to the phylum Heterokontophyta include algae belonging to the class Chrysophyceae, Dictyochophyceae, Pelagophyceae, Rhaphidophyceae, Bacillariophyceae, Phaeophyceae, Xanthophyceae, Eustigmatophyceae, or the like. Examples of algae belonging to the class Bacillariophyceae include Thalassiosira algae such as Thalassiosira pseudonana (Tonon, T. et al., 2002, Phytochemistry, 61:15-24).

Specific examples of Neochloris oleoabundans include the Neochloris oleoabundans UTEX 1185 strain. Specific examples of Nannochloris sp. include the Nannochloris sp. UTEX LB 1999 strain. Specific examples of Chlorella kessleri include the Chlorella kessleri 11h strain (UTEX 263). Specific examples of Thalassiosira pseudonana include the Thalassiosira pseudonana UTEX LB FD2 strain. These strains can be obtained from the University of Texas at Austin, The Culture Collection of Algae (UTEX), 1 University Station A6700, Austin, Tex. 78712-0183, USA.

Microalga belonging to the class Chlorophyceae, Trebouxiophyceae, or Bacillariophyceae are particular examples, and algae belonging to the class Chlorophyceae is a particular example.

Labyrinthulea, which refer to fungus-like unicellular protests that accumulate highly unsaturated fatty acids such as DHA (docosahexaenoic acid) and EPA (eicosapentaenoic acid) at high concentration, may also be classified as microalgae. Labyrinthulea specifically includes the genera Aurantiochytrium, Schizochytrium, Thraustochytrium, Ulkenia, and so forth. Although Labyrinthulea does not perform photosynthesis, and hence are cultured under heterotrophic conditions, the method as described herein can be applied thereto.

Any medium typically employed in culture of a microalga can be used in the method described herein. There are many findings concerning culture of microalgae, and Chlorella algae, Arthrospira algae (Spirulina), Dunaliella salina, and so forth have been industrially cultured in a large scale for edible use (Spolaore, P. et al., 2006, J. Biosci. Bioeng., 101:87-96). For the culture of Chlamydomonas reinhardtii, for example, the 0.3×HSM medium (Oyama, Y. et al., 2006, Planta 224:646-654) can be used. For the culture of Chlorella kessleri, the 0.2×Gamborg's medium (Izumo, A. et al., 2007, Plant Science 172:1138-1147) and so forth can be used. Neochloris oleoabundans and Nannochloris sp. can be cultured by using the modified NORO medium (Yamaberi, K. et al., 1998, J. Mar. Biotechnol., 6:44-48; Takagi, M. et al., 2000, Appl. Microbiol. Biotechnol., 54:112-117) and the Bold's Basal Medium (Tornabene, T. G. et al., 1983, Enzyme and Microb. Technol., 5:435-440; Archibald, P. A. and Bold, H. C., 1970, Phytomorphology, 20:383-389). As for the algae belonging to the class Bacillariophyceae, for the culture of Thalassiosira pseudonana, the F/2 medium (Lie, C.-P. and Lin, L.-P., 2001, Bot. Bull. Acad. Sin., 42:207-214) and so forth can be used.

Further, it is known that algae accumulate fats and oils in alga bodies when the nitrogen source is depleted (Thompson G A Jr., 1996, Biochim. Biophys. Acta, 1302:17-45). A medium containing a limited concentration of a nitrogen source can also be used for the culture of the microalga.

Culture systems for microalgae include open-type culture systems called open ponds and closed-type culture systems called closed photobioreactors, and any of these culture systems can be used. Furthermore, culture conditions for microalgae can include autotrophic culture, which depends only on photosynthesis; heterotrophic culture, which depends on a carbon source; and mixotrophic culture, which uses both photosynthesis and an organic compound. Any of these culture conditions can be used.

The culture can be performed by adding 1 to 50% by volume of precultured broth based on the volume of the medium of main culture in many cases. Initial pH of the medium can be around neutral. “pH around neutral” may be, for example, pH 7 to 9. Although pH adjustment is not performed during culture in many cases, pH may be adjusted if needed. The culture temperature can be 25 to 35° C., and in particular, a temperature around 28° C. is generally frequently used. However, the culture temperature may be any temperature suitable for the alga to be cultured. Air can be added into the culture medium. An aeration rate can be an aeration volume per unit volume of culture medium per minute of, for example, 0.1 to 2 vvm (volume per volume per minute). Furthermore, CO₂ may also be added into the culture medium. It is expected that adding CO₂ accelerates growth of the microalga. CO₂ can be added at about 0.5 to 5% of the aeration rate. When culture using photosynthesis is performed, light is irradiated in the culture system. Since optimum illumination intensity of light differs depending on type of microalgae, light may be irradiated at an illumination intensity suitable for the microalga to be cultured. An illumination intensity of about 1,000 to 10,000 luxes is frequently used. As the light source, a white fluorescent lamp is commonly used indoors, but the light source is not limited thereto. As the light source, it is also possible to use sunlight outdoors. The culture medium may be stirred or circulated at an appropriate intensity, if needed. Although the culture period is not particularly limited, it may be, for example, 1 to 40 days.

By culturing a microalga as described above, alga bodies of the microalga are produced in the medium.

<1-2> Biomass Derived from Microalga

Examples of the “biomass derived from a microalga” (henceforth also referred to as “microalga biomass” or simply referred to as “biomass”) can include the alga bodies themselves of a cultured microalga, and can also include a treated or processed product of the alga bodies. Examples of the treated product of alga bodies of a microalga can include, but are not particularly limited to, a disruption product of alga bodies of a microalga, as well as the residue that remains after extracting a desired ingredient from alga bodies of a microalga (also referred to as “alga body residue” or “residual alga bodies”). Examples of the desired ingredient include effective ingredients such as bio-fuels. Examples of the effective ingredients such as bio-fuels include fatty acids, hydrolysates of fats and oils, lipids such as terpenoids and steroids, and hydrocarbons.

In the method for producing a nutrient additive, the expression “culturing a microalga in a medium to produce biomass derived from the microalga” may mean to culture a microalga in a medium thereby to produce alga bodies of the microalga as the biomass in the medium, when alga bodies themselves of the microalga are used as the biomass, or may mean to culture a microalga in a medium and treat or process alga bodies of the microalga produced by the culture thereby to produce a treated product of the alga bodies, when the treated product of the alga bodies of the microalga is used as the biomass.

When alga bodies of a microalga are subjected to such a treatment or process such as disruption and extraction, the treatment may occur when the alga bodies are in the medium, after they have been diluted or concentrated, or after they have been collected from the medium.

Examples of the method for collecting the alga bodies from the culture medium can include typical methods such as centrifugation, filtration, or gravitational precipitation using a flocculant (Grima, E. M. et al., 2003, Biotechnol. Advances, 20:491-515). That is, the alga bodies can be precipitated spontaneously or by using a flocculant, and the precipitated alga bodies can be collected. The alga bodies can also be precipitated by centrifugation, and the precipitated alga bodies can be collected. Furthermore, for example, by precipitating alga bodies, and appropriately removing the supernatant, the alga bodies can be concentrated to a desired extent. Furthermore, alga bodies can be diluted to a desired extent by using an arbitrary medium, for example, an aqueous medium such as water or a buffer.

The desired ingredient can be extracted from the alga bodies by a method appropriately chosen according to the type of the microalga, type of the ingredient, etc. When a residue obtained after extracting the desired ingredient from alga bodies of a microalga is used as the microalga biomass, for example, the residue can be obtained by disrupting alga bodies one time and extracting effective ingredients such as fats and oils, or the residue can be obtained by treating alga bodies at a moderate temperature to generate fats and oils (described in WO2011/013707).

As the method for disrupting alga bodies, there are various methods for various uses, and any of such methods may be used. As the method for disrupting alga bodies, for example, a high temperature treatment, organic solvent treatment, boiling treatment, strong alkali treatment, ultrasonic treatment, French press treatment, or any combination thereof can be used. Examples of the high temperature treatment include, for example, a treatment at a temperature of 100° C. or higher, 150° C. or higher, or 175 to 215° C. The high temperature treatment can include a high temperature and high pressure reaction under the conditions for a reaction called hydrothermal reaction. Examples of the organic solvent treatment can include, for example, a treatment with a mixed solvent of methanol and chloroform. Furthermore, alga bodies may be dried, and then physically disrupted. Extraction efficiency of a fat-soluble substance can generally be improved by disrupting the alga bodies. After the alga bodies are disrupted, fat-soluble effective ingredients such as bio-fuels can be extracted from the disruption product by solvent extraction. For example, fats and oils can be extracted from the disruption product of alga bodies by adding 80% methanol or 80% acetone to the disruption product of alga bodies, and extracting the fats and oils not dissolved in the foregoing solvent with another solvent such as hexane and chloroform, the fats and oils can be extracted as a crude fat-soluble fraction.

Examples of the method for treating the alga bodies of microalga also can include a treatment at a moderate temperature as described in WO2011/013707 (henceforth also referred to as “moderate temperature treatment”). Specifically, for example, alga bodies are treated at a moderate temperature, the product of the treatment is separated into precipitate and supernatant by centrifugation, fats and oils produced by the microalga and present in the precipitate are extracted with an organic solvent, and then the remaining residue can be used as the microalga biomass. Although the residue can be used as it is, it can also be concentrated by lyophilization or evaporation, or the like.

A moderate temperature can mean a temperature sufficient for increasing the quantity of a fatty acid, glycerol, or glucose in the treated product. For example, the alga bodies may be continuously treated at a constant temperature (henceforth referred to as “continuous moderate temperature treatment”), or may be treated while the temperature is lowered during the treatment. Examples of the embodiment in which the temperature is lowered during the treatment can include embodiments in which the alga bodies are treated once at a moderate temperature as the first moderate temperature treatment, and then further treated at a certain temperature lower than the temperature of the first moderate temperature treatment as the second moderate temperature treatment. The continuous moderate temperature treatment and the first moderate temperature treatment are usually performed at a temperature of 40° C. or higher, 45° C. or higher, 50° C. or higher, as for the lowest temperature, and usually performed at a temperature of 70° C. or lower, 65° C. or lower, 60° C. or lower, as for the highest temperature. The second moderate temperature treatment is usually performed at 30° C. or higher, 35° C. or higher, 40° C. or higher, as for the lowest temperature, and usually performed at 55° C. or lower, 50° C. or lower, 45° C. or lower, as for the highest temperature.

For the moderate temperature treatment, culture broth containing the alga bodies obtained by the aforementioned method for culturing alga may be used as it is, or a fraction containing the alga bodies may be appropriately concentrated and used. For example, collected alga bodies may be used for the moderate temperature treatment.

Furthermore, before the moderate temperature treatment, pH of the reaction system may be adjusted to be a weakly acidic pH, and/or the alga bodies may be once frozen.

The weakly acidic pH mentioned above may be 3.0 to 7.0, or 4.0 to 6.0.

The temperature for freezing the alga bodies may be usually a temperature not lower than −80° C. and not higher than 0° C., not higher than −20° C., or not higher than −50° C. The period for freezing the alga bodies can be 1 hour or longer.

The period for the continuous moderate temperature treatment may be at least 1 hour or longer, or 5 hours or longer. The period for the continuous moderate temperature treatment may usually be 48 hours or shorter, or 24 hours or shorter. Furthermore, the period for the first step moderate temperature treatment may be at least 1 minute or longer, 10 minutes or longer, or 20 minutes or longer. The period for the first step moderate temperature treatment may usually be 120 minutes or shorter, or 60 minutes or shorter. Furthermore, the period for the second step moderate temperature treatment may be at least 1 hour or longer, or 4 hours or longer. The period for the second step moderate temperature treatment may usually be 20 hours or shorter, or 15 hours or shorter.

After the moderate temperature treatment, an alkaline treatment or an organic solvent treatment may be further performed. When an alkaline treatment or an organic solvent treatment is performed after the moderate temperature treatment, the treated product obtained after the moderate temperature treatment may be subjected to the treatment (alkaline treatment or an organic solvent treatment) as it is or after dilution, or a fraction containing biomass may be appropriately concentrated and subjected to the treatment. The expression “a fraction containing biomass is concentrated and subjected to the treatment” can include, for example, concentrating biomass present in the product of the moderate temperature treatment to a desired extent, and subjecting the concentrated product to the above described alkaline or organic solvent treatment, and also separating the precipitate from the supernatant after concentrating the biomass, and subjecting the separated precipitate to the treatment. The concentration of the precipitate (solid content) in the reaction mixture in which the alkaline treatment or organic solvent treatment is performed may be, for example, 250 g/L or lower, 125 g/L or lower. In the case of the alkaline treatment, the reaction mixture in which the concentration of the precipitate (solid content) is 125 g/L or lower can be subjected to the treatment. In the case of the organic solvent treatment, the precipitate separated from the supernatant can be subjected to the treatment.

The pH for the alkaline treatment to be performed after the moderate temperature treatment can be not lower than 10.5 and not higher than 14, not lower than 11.5, or not lower than 12.5. For the alkaline treatment, an alkaline substance such as NaOH or KOH can be used.

The temperature for the alkaline treatment may be 60° C. or higher, 80° C. or higher, or 90° C. or higher. The temperature for the alkaline treatment can be 120° C. or lower.

The period for the alkaline treatment may be at least 10 minutes or longer, 30 minutes or longer, or 60 minutes or longer. The period for the alkaline treatment can be 150 minutes or shorter.

As the organic solvent treatment to be performed after the moderate temperature treatment, the treated product of the moderate temperature treatment may be dried and subjected to the organic solvent treatment, or the treated product of the moderate temperature treatment may be subjected to the organic solvent treatment without drying. Examples of the organic solvent can include methanol, ethanol, 2-propanol, acetone, butanol, pentanol, hexanol, heptanol, octanol, chloroform, methyl acetate, ethyl acetate, dimethyl ether, diethyl ether, hexane, and so forth.

<1-3> Acid Hydrolysis of Biomass Derived from Microalga and Preparation of Nutrient Additive

In the method for producing a nutrient additive, biomass derived from a microalga is hydrolyzed by adding an acid to the biomass.

The acid may be added to the biomass derived from a microalga per se, or to a fraction containing the biomass derived from a microalga. The “biomass derived from a microalga per se” can mean collected biomass, for example, alga bodies collected from a medium, or a treated product of alga bodies such as an alga body disruption product or an alga body residue collected from any of various kinds of treatment mixtures. The “fraction containing biomass derived from a microalga” can mean an arbitrary fraction containing biomass, for example, a culture broth containing alga bodies, a treatment mixture containing a treated product of alga bodies such as an alga body disruption product or an alga body residue, or a diluted or concentrated product of such a treatment mixture. That is, the biomass derived from a microalga may be subjected to the hydrolysis treatment while contained in the medium or any of various treatment mixtures, after diluted or concentrated, or after collected from the medium or any of various treatment mixtures. The biomass may be diluted, concentrated, or collected in the same manner as that for the dilution, concentration, or collection of alga bodies mentioned above. In the method for producing a nutrient additive, only one kind of biomass may be used, or a combination of two or more kinds of biomasses may be used. The expression of “adding an acid to biomass” shall also include embodiments in which the biomass and the acid are mixed, and embodiments in which the biomass is added to the acid.

The acid used for the hydrolysis may be any acid that hydrolyzes the biomass derived from a microalga. As the acid, in particular, sulfuric acid, hydrochloric acid, nitric acid, and phosphoric acid can be used.

The ratio of the total amount of solid content of the biomass derived from a microalga to the total amount of the reaction mixture in which the hydrolysis is performed may be 5 to 80% (w/w), or 10 to 40% (w/w). The amount of the acid to be added can be adjusted so that the molar ratio of the anions thereof to nitrogen contained in the alga body biomass is 0.1 to 100, 0.1 to 20, or 0.1 to 10. The amount of the acid to be added may be appropriately changed depending on the type of the acid, and so forth.

For example, when sulfuric acid is used, it can be added in such an amount that the molar ratio of sulfate ions to nitrogen contained in the alga body biomass (SO₄/N) is 0.1 to 100, 0.1 to 10, or 0.8 to 3. As sulfuric acid, 98% sulfuric acid can be used, but sulfuric acid is not limited thereto.

Also when an acid other than sulfuric acid is used, the hydrolysis treatment can be performed under appropriately chosen conditions where the biomass derived from a microalga can be hydrolyzed. When an acid other than sulfuric acid is used, the hydrolysis treatment may be performed under the same conditions as when using sulfuric acid, or the conditions may be appropriately changed according to the type of the acid, and so forth. When an acid other than sulfuric acid is used, the term “sulfate ions” in the aforementioned explanation may include corresponding to the acid to be used.

For example, when hydrochloric acid is used, hydrochloric acid may be added in such an amount that the molar ratio of chloride ions to nitrogen contained in the alga body biomass (Cl/N) is 0.1 to 100, 0.1 to 20, or 0.8 to 3.

For example, when phosphoric acid is used, phosphoric acid may be added in such an amount that the molar ratio of phosphate ions to nitrogen contained in the alga body biomass (PO₄/N) is 0.1 to 100, 0.1 to 50, or 0.8 to 20.

For example, when nitric acid is used, nitric acid may be added in such an amount that the molar ratio of nitrate ions to nitrogen contained in the alga body biomass (NO₃/N) is 0.1 to 100, 0.1 to 50, or 0.8 to 20.

After the acid is added, the reaction mixture is treated at 75 to 130° C., or 110 to 120° C., for 5 to 50 hours, or for 10 to 32 hours. Before the acid treatment, the biomass may be treated at 80 to 110° C., or 90 to 105° C., for 30 minutes to 2 hours, or 40 minutes to 90 minutes.

After the hydrolysis is performed, a treatment for neutralizing the reaction mixture may be performed. The reaction mixture can be neutralized by adding an alkali to the reaction mixture. Examples of alkali can include, but are not particularly limited to, NaOH and KOH. pH of the reaction mixture after the neutralization may be, for example, 5 to 7.

After the hydrolysis is performed, insoluble matter can be removed from the reaction mixture by, for example, filtration or centrifugation.

As described above, a hydrolysate of the biomass derived from a microalga can be obtained.

In the method for producing a nutrient additive, the hydrolysate of the biomass is prepared as a nutrient additive. The expression that “the hydrolysate of the biomass is prepared as a nutrient additive” can mean that the nutrient additive is prepared by using the hydrolysate of the biomass obtained as described above as an effective ingredient. The nutrient additive may consist of the hydrolysate of the biomass derived from a microalga, or may also contain other ingredients. That is, the expression that “the hydrolysate of the biomass is prepared as a nutrient additive” can mean that the hydrolysate of the biomass obtained as described above is used as it is as the nutrient additive, or the hydrolysate of the biomass obtained as described above is combined with another ingredient to prepare the nutrient additive. The hydrolysate of the biomass may be appropriately diluted or concentrated, and then used for the preparation of the nutrient additive. The other ingredient is not particularly limited, so long as it is an ingredient that can be used for culturing a microorganism or microalga.

As described above, the nutrient additive can be obtained.

The hydrolysate of the biomass derived from a microalga and the nutrient additive containing the hydrolysate as an effective ingredient can promote the growth of a microorganism or microalga, or can promote production of a substance by a microorganism or microalga.

The expression that “growth of a microorganism or microalga is promoted” can mean that growth of the microorganism or microalga is promoted when the microorganism or microalga is cultured in a medium containing the hydrolysate or the nutrient additive, as compared with that observed when the microorganism or microalga is cultured in a medium not containing the hydrolysate or the nutrient additive, while the degree of the promotion is not particularly limited. The expression that “growth of a microorganism or microalga is promoted” can mean that growth of the microorganism or microalga is promoted by 5% or more, 10% or more, or 20% or more when the microorganism or microalga is cultured in a medium containing the hydrolysate or the nutrient additive, as compared with that observed when the microorganism or microalga is cultured in a medium not containing the hydrolysate or the nutrient additive. Growth of a microorganism or microalga can be measured by measuring OD value or dry alga body weight.

Furthermore, the expression that “production of a substance by the microorganism or microalga is promoted” can mean that production of an objective substance by the microorganism or microalga is improved when the microorganism or microalga is cultured in a medium containing the hydrolysate or the nutrient additive, as compared with that observed when the microorganism or microalga is cultured in a medium not containing the hydrolysate or the nutrient additive, while the degree of the promotion is not particularly limited. The expression that “production of a substance by the microorganism or microalga is promoted” may mean that production of an objective substance by the microorganism or microalga is improved by 1% or more, 5% or more, or 10% or more when the microorganism or microalga is cultured in a medium containing the hydrolysate or the nutrient additive, as compared with that observed when the microorganism or microalga is cultured in a medium not containing the hydrolysate or the nutrient additive. The “improvement of the production of an objective substance” referred to above may be improvement in production amount, productivity, and/or yield of the objective substance.

Whether the hydrolysate or the nutrient additive promotes growth of a microorganism or microalga or production of a substance can be confirmed by culturing the microorganism or microalga with and without addition of the hydrolysate or the nutrient additive under the same conditions, and comparing the amount of growth of the microorganism or microalga or the amount of production of the substance by the microorganism or microalga.

The amount of the hydrolysate present in the nutrient additive is not particularly limited so long as the nutrient additive is able to promote growth of a microorganism or microalga, or promote production of a substance by a microorganism or microalga. The amount of the hydrolysate present in the nutrient additive may be, for example, so that when the nutrient additive is added to a medium, the concentration of the hydrolysate in the medium is 1 mM to 100 mM, or 10 mM to 30 mM, in terms of the nitrogen amount.

<2> Use of Nutrient Additive

The nutrient additive can be used for culturing a microorganism or microalga.

That is, the presently described subject matter provides a method for culturing a microalga or a microorganism including the steps of culturing a microalga or a microorganism in a medium containing the nutrient additive (henceforth also referred to as the “culture method of the present invention”). In one of many numerous embodiments, a microalga or a microorganism can be cultured to produce a desired objective substance. That is, an embodiment of the culture method may be a method for producing an objective substance by culturing a microalga or a microorganism in a medium containing the nutrient additive.

In the culture method, a microorganism or a microalga can be cultured under conditions typically used for culturing a microorganism or microalga, or conditions typically used for culturing a typical microorganism or microalga to produce an objective substance, except that a medium containing the nutrient additive as described herein is used.

The microalga can be cultured, for example, under the same conditions as those for the culture of a microalga in the method for producing a nutrient additive as described above, except that the medium containing the nutrient additive is used. Specifically, any media that can be used for culture of a microalga, such as the 0.2×Gamborg's B5 medium, which is used in the Examples, BG11 medium, and AF-6 medium, can be used, after the nutrient additive has been added.

A microorganism can be cultured in, for example, an appropriate medium containing a carbon source, nitrogen source, sulfur source, and inorganic ions, as well as other organic components as required, and the nutrient additive as described herein. Further, antibiotics and/or a gene expression inducer can also be added to the medium, as required. Examples of the carbon source include, for example, saccharides such as glucose, fructose, sucrose, molasses, and hydrolysate of starch, alcohols such as glycerol and ethanol, and organic acids such as fumaric acid, citric acid, and succinic acid. Examples of the nitrogen source include, for example, inorganic ammonium salts such as ammonium sulfate, ammonium chloride, and ammonium phosphate, organic nitrogens such as soybean hydrolysate, ammonia gas, and aqueous ammonia. Examples of the sulfur source include, for example, inorganic sulfur compounds such as sulfates, sulfites, sulfides, hyposulfites, and thiosulfates. Examples of the inorganic ions include, for example, calcium ion, magnesium ion, phosphate ion, potassium ion, and iron ion. Examples of the other organic components include organic micronutrients. As the organic micronutrients, for example, vitamins, amino acids, yeast extract containing them, and so forth can be used. The culture can be performed, for example, under aerobic conditions for 12 to 100 hours. The culture temperature may be, for example, 25 to 40° C. The culture pH may be controlled to be, for example, 5 to 8. To adjust the pH, inorganic or organic acidic or alkaline substances, as well as ammonia gas, aqueous ammonia, and so forth can be used.

Furthermore, it is also possible to reduce the amount(s) of ingredient(s) in existing media, for example, such as the carbon source, nitrogen source, phosphoric acid source, sulfur source, and potassium source, by adding the nutrient additive to the media. The direct cost of the medium can be thereby reduced.

The amount of the nutrient additive to be added is not particularly limited, so long as the effect of the nutrient additive can be obtained. The expression that “the effect of the nutrient additive can be obtained” can mean that the effect of promoting growth of a microorganism or microalga, or the effect of promoting production of a substance by a microorganism or microalga can be obtained. An appropriate amount of the nutrient additive to be added can be determined by, for example, culturing a microorganism or microalga in media containing the nutrient additive at various concentrations, and comparing the degrees of growth or production of a substance. The amount of the nutrient additive to be added may be such an amount that the concentration of the nutrient additive in the medium is 1 mM to 100 mM, or 10 mM to 30 mM, in terms of the nitrogen amount.

In the culture method as described herein, one kind of the nutrient additive may be added to the medium, or two or more kinds of the nutrient additives may be added to the medium. For example, an acid hydrolysis product of alga bodies and an acid hydrolysis product of an alga body residue may be added to the medium in combination. When two or more kinds of the nutrient additives are added to the medium, the combination ratio of them is not particularly limited so long as the effect of the nutrient additive can be obtained.

The nutrient additive may be added to the medium at or before the start of the culture, or after the start of the culture. That is, the expression “culturing a microalga or a microorganism in a medium containing the nutrient additive” may also include when a medium without the nutrient additive is used during a part of the culture period. The “part of the culture period” referred to above may be, for example, 10% or less, 20% or less, or 30% or less, of the total culture period. In addition, the nutrient additive may be added to the medium continuously or intermittently. When added continuously, the continuous addition may be over the total culture period, or over a part of the culture period. Furthermore, during continuous addition, the addition rate and/or type of the nutrient additive may be fixed, or may not be fixed, over the total period of the addition. Furthermore, when the nutrient additive is intermittently added to the medium two or more times, the addition amount and/or type of the nutrient additive may be the same, or may not be the same, for every addition.

The microalga to be cultured by the culture method as described herein may be any of the microalgae described above.

Furthermore, the microorganism to be cultured by the culture method as described herein may be any microorganism. Examples of the microorganism include bacteria, and for example, coryneform bacteria, Bacillus bacteria, and bacteria belonging to the family Enterobacteriaceae are particular examples.

The coryneform bacteria are aerobic, GC-rich gram-positive bacilli. The coryneform bacteria include bacteria that have previously been classified into the genus Brevibacterium but are currently united into the genus Corynebacterium (Int. J. Syst. Bacteriol. 41:255 (1991)), and bacteria belonging to the genus Brevibacterium, which are closely related to the genus Corynebacterium.

Examples of the coryneform bacteria include the followings species.

Corynebacterium acetoacidophilum

Corynebacterium acetoglutamicum

Corynebacterium alkanolyticum

Corynebacterium callunae

Corynebacterium glutamicum (Brevibacterium lactofermentum)

Corynebacterium lilium

Corynebacterium melassecola

Corynebacterium thermoaminogenes

Corynebacterium herculis

Brevibacterium divaricatum

Brevibacterium flavum

Brevibacterium immariophilum

Brevibacterium lactofermentum

Brevibacterium roseum

Brevibacterium saccharolyticum

Brevibacterium thiogenitalis

Corynebacterium ammoniagenes (Corynebacterium stationis)

Brevibacterium album

Brevibacterium cerinum

Microbacterium ammoniaphilum

Specific examples of the coryneform bacteria include the following strains.

Corynebacterium acetoacidophilum ATCC 13870

Corynebacterium acetoglutamicum ATCC 15806

Corynebacterium alkanolyticum ATCC 21511

Corynebacterium callunae ATCC 15991

Corynebacterium glutamicum (Brevibacterium lactofermentum) ATCC 13020, ATCC 13032, ATCC 13060, ATCC 13869, FERM BP-734

Corynebacterium lilium ATCC 15990

Corynebacterium melassecola ATCC 17965

Corynebacterium efficiens AJ12340 (FERM BP-1539)

Corynebacterium herculis ATCC 13868

Brevibacterium divaricatum ATCC 14020

Brevibacterium flavum ATCC 13826, ATCC 14067, AJ12418 (FERM BP-2205)

Brevibacterium immariophilum ATCC 14068

Brevibacterium lactofermentum ATCC 13869

Brevibacterium roseum ATCC 13825

Brevibacterium saccharolyticum ATCC 14066

Brevibacterium thiogenitalis ATCC 19240

Corynebacterium ammoniagenes (Corynebacterium stationis) ATCC 6871, ATCC 6872

Brevibacterium album ATCC 15111

Brevibacterium cerinum ATCC 15112

Microbacterium ammoniaphilum ATCC 15354

The Enterobacteriaceae bacteria are not particularly limited so long as they belong to the family Enterobacteriaceae such as those of the genera Escherichia, Enterobacter, Pantoea, Klebsiella, Serratia, Erwinia, Salmonella, and Morganella. Specifically, those classified into the family Enterobacteriaceae according to the taxonomy used in the NCBI (National Center for Biotechnology Information) database (www.ncbi.nlm.nih.gov/htbin-post/Taxonomy/wgetorg?mode=Tree&id=1236&1v1=3&keep=1&srchmode=1&unlock) can be used. As the Enterobacteriaceae bacteria, bacteria of the genus Escherichia is a particular example.

Although the Escherichia bacteria are not particularly limited, specifically, those described in the work of Neidhardt et al. (Backmann B. J., 1996, Derivations and Genotypes of some mutant derivatives of Escherichia coli K-12, p. 2460-2488, Table 1, In F. D. Neidhardt (ed.), Escherichia coli and Salmonella Cellular and Molecular Biology/Second Edition, American Society for Microbiology Press, Washington, D.C.) can be used. Examples of Escherichia bacteria include, for example, Escherichia coli. Specific examples of Escherichia coli include strains derived from the Escherichia coli K12 strain, such as the Escherichia coli MG1655 strain (ATCC 47076) and the Escherichia coli W3110 strain (ATCC 27325).

The aforementioned strains with ATCC numbers are available from, for example, the American Type Culture Collection (Address: P.O. Box 1549, Manassas, Va. 20108, United States of America). That is, registration numbers are given to the respective strains, and the strains can be ordered by using these registration numbers (refer to http://www.atcc.org/). The registration numbers of the strains are listed in the catalogue of the American Type Culture Collection.

Examples of the Enterobacter bacteria include Enterobacter agglomerans, Enterobacter aerogenes, and so forth, and examples of the Pantoea bacteria include Pantoea ananatis. Some strains of Enterobacter agglomerans were recently reclassified into Pantoea agglomerans, Pantoea ananatis, or Pantoea stewartii on the basis of nucleotide sequence analysis of 16S rRNA etc. A bacterium to be used may belong to either genus Enterobacter or Pantoea so long as it is a bacterium classified into the family Enterobacteriaceae. When Pantoea ananatis is bred by using genetic engineering techniques, for example, the Pantoea ananatis AJ13355 strain (FERM BP-6614), AJ13356 strain (FERM BP-6615), AJ13601 strain (FERM BP-7207), and derivatives thereof can be used as parent strains. These strains were identified and deposited as Enterobacter agglomerans when they were isolated, but as described above, these strains have been reclassified as Pantoea ananatis on the basis of the nucleotide sequence analysis of 16S rRNA etc.

Examples of Bacillus bacteria include Bacillus subtilis such as Bacillus subtilis 168 Marburg strain (ATCC 6051).

When an objective substance is produced by the culture method as described herein, a microorganism or microalga having an ability to produce the objective substance is used. The objective substance to be produced by the culture method may be any substance. When using a microorganism, examples of the objective substance include L-amino acids and nucleic acids. When using a microalga, examples of the objective substance include starches, hydrolysates of starches (also referred to as saccharification products of starches), fatty acids, hydrolysates of fats and oils, lipids, and hydrocarbons. In the culture method, one kind of objective substance may be produced, or two or more kinds of objective substances may be produced.

In the culture method, a microorganism or microalga may produce an objective substance itself, or a substance produced by a microorganism or microalga may be further treated to produce an objective substance. That is, the culture method may include the step of further treating a substance produced by a microorganism or microalga thereby to produce an objective substance. When a substance produced by a microorganism or microalga is further treated to produce an objective substance, the “ability to produce an objective substance” may mean an ability to produce a substance that is changed into the objective substance by such a treatment. Specifically, for example, when the objective substance is a hydrolysate of starch, the “ability to produce an objective substance” may mean an ability to produce starch.

A microorganism or microalga having an ability to produce an objective substance can be obtained by, for example, a known method. The microorganism or microalga having an ability to produce an objective substance may be a microorganism or microalga inherently having the ability to produce the objective substance, or a microorganism or microalga to which the ability to produce the objective substance is imparted or enhanced.

An L-amino acid-producing bacterium can be obtained by, for example, imparting an L-amino acid-producing ability to such a bacterium as mentioned above, or by enhancing an L-amino acid-producing ability of such a bacterium as mentioned above.

To impart or enhance an L-amino acid-producing ability, methods conventionally employed in the breeding of amino acid-producing strains of coryneform bacteria, Escherichia bacteria, and so forth (see “Amino Acid Fermentation”, Gakkai Shuppan Center (Ltd.), 1st Edition, published May 30, 1986, pp. 77-100) can be used. Examples of such methods include, for example, acquiring an auxotrophic mutant strain, acquiring an L-amino acid analogue-resistant strain, acquiring a metabolic regulation mutant strain, and constructing a recombinant strain in which the activity of an L-amino acid biosynthetic enzyme is enhanced. In the breeding of L-amino acid-producing bacteria, one of the above-described properties such as auxotrophy, analogue resistance, and metabolic regulation mutation may be imparted alone, or two or three or more of such properties may be imparted in combination. Also, in the breeding of L-amino acid-producing bacteria, the activity of an L-amino acid biosynthetic enzyme may be enhanced alone, or the activities of two or three or more of such enzymes may be enhanced in combination. Furthermore, imparting property(s) such as auxotrophy, analogue resistance, and metabolic regulation mutation can be combined with enhancing the activity(s) of biosynthetic enzyme(s).

An auxotrophic mutant strain, L-amino acid analogue-resistant strain, or metabolic regulation mutant strain having an L-amino acid-producing ability can be obtained by subjecting a parent strain or wild-type strain to a usual mutagenesis treatment, and then selecting a strain exhibiting autotrophy, analogue resistance, or a metabolic regulation mutation, and having an L-amino acid-producing ability from the obtained mutant strains. Examples of the usual mutagenesis treatment include irradiation of X-ray or ultraviolet and a treatment with a mutation agent such as N-methyl-N′-nitro-N-nitrosoguanidine.

Furthermore, an L-amino acid-producing ability can also be imparted or enhanced by enhancing the activity of an enzyme involved in biosynthesis of the objective L-amino acid. An enzyme activity can be enhanced by, for example, modifying a bacterium so that the expression of a gene coding for the enzyme is enhanced. Methods for enhancing gene expression are described in WO00/18935, EP 1010755 A, and so forth.

Furthermore, an L-amino acid-producing ability can also be imparted or enhanced by reducing the activity of an enzyme that catalyzes a reaction causing a branching away from the biosynthetic pathway of the objective L-amino acid and resulting in generation of a compound other than the objective L-amino acid.

A microorganism or microalga having an ability to produce another objective substance such as a nucleic acid can also be obtained by the same method as that for obtaining an L-amino acid-producing bacterium.

Examples of the L-amino acid include L-lysine, L-ornithine, L-arginine, L-histidine, L-citrulline, L-isoleucine, L-alanine, L-valine, L-leucine, glycine, L-threonine, L-serine, L-proline, L-phenylalanine, L-tyrosine, L-tryptophan, L-cysteine, L-cystine, L-methionine, L-glutamic acid, L-aspartic acid, L-glutamine, and L-asparagine.

Examples of the nucleic acid include purine nucleosides, purine nucleotides, and so forth. Examples of the purine nucleosides include inosine, xanthosine, guanosine, adenosine, and so forth. Examples of the purine nucleotides include 5′-phosphate esters of the purine nucleosides, such as inosinic acid (inosine-5′-phosphate, henceforth also referred to as “IMP”), xanthylic acid (xanthosine-5′-phosphate, henceforth also referred to as “XMP”), guanylic acid (guanosine-5′-monophosphate, henceforth also referred to as “GMP”), adenylic acid (adenosine-5′-monophosphate, henceforth also referred to as “AMP”), and so forth.

Starch is a high molecular weight polysaccharide consisting of amylose, which consists of glucose residues linearly linked by α-1,4-glycoside linkages, and amylopectin, which has a branched structure consisting of glucose residues linearly linked by α-1,4-glycoside linkages and α-1,6-glycoside linkages. Amylase is a generic name of enzymes that hydrolyze glycoside linkages of starch etc. Amylases are roughly classified into α-amylase (EC 3.2.1.1), β-amylase (EC 3.2.1.2), and glucoamylase (EC 3.2.1.3) according to the difference in sites on which amylases act. α-Amylase is an endo-type enzyme which randomly cleaves α-1,4-glycoside linkages of starch, glycogen, and so forth. β-Amylase is an exo-type enzyme which cleaves α-1,4-glycoside linkage to excise maltose units one by one from the non-reducing end of starch. The glucoamylase (also called amyloglucosidase) is an exo-type enzyme which cleaves α-1,4-glycoside linkages to excise glucose units one by one from the non-reducing end of starch, and also cleaves α-1,6-glycoside linkages in amylopectin. Since glucoamylase produces glucose directly from starch, it is widely used for the production of glucose, and it is a exemplary enzyme in the methods described herein.

There are many industrially implemented examples of saccharification reactions of starch derived from grains (Robertson, G. H. et al., J. Agric. Food Chem., 54:353-365, 2006). In the same manner as those used in these examples, a saccharification product of starch can be obtained from alga bodies containing starch by an enzymatic reaction. It is also possible to disrupt alga bodies containing starch, and enzymatically treat a suspension containing disrupted alga bodies thereby to obtain a saccharification product of starch. When a suspension containing disrupted alga bodies is subjected to an enzyme treatment, it is preferable to perform boiling, ultrasonication, alkaline treatment, or the like in combination, as a pretreatment (Izumo A. et al., Plant Science, 172:1138-1147, 2007).

Conditions for the enzymatic reaction can be appropriately determined according to the characteristics of the enzyme to be used. For example, for amyloglucosidase (Sigma Aldrich, A-9228), the following conditions are exemplary: an enzyme concentration of 2 to 20 U/mL, a temperature of 40 to 60° C., and pH 4 to 6.

The saccharification product of starch can be used as, for example, a carbon source for culturing a bacterium such as an L-amino acid-producing bacterium. Therefore, if an organic acid that can be utilized by a bacterium is used as a buffer for adjusting pH at the time of the enzymatic reaction (at the time of saccharification), the organic acid can be used as a carbon source for culturing the bacterium together with the saccharification product of starch. For example, the enzyme reaction product as it is can be added to the medium.

The saccharification product of starch produced by a microalga refers to a product obtained by hydrolysis of starch, in which oligosaccharides or monosaccharides that can be assimilated by bacteria, such as maltose or glucose, have been thereby generated. In the saccharification product of starch produced by a microalga, starch may be substantially totally saccharified, or may be partially saccharified. The saccharification product of starch produced by a microalga may be a saccharification product in which 50% by weight or more, 70% by weight or more, or 90% by weight or more, of starch is converted into glucose. Furthermore, the saccharification product of starch produced by a microalga may contain carbohydrates produced by the microalga other than starch or a saccharification product thereof.

Fats and oils are esters of fatty acid(s) and glycerol, and are also called triglycerides. Fatty acids can be used as, for example, a carbon source for culturing a bacterium such as an L-amino acid-producing bacterium. Therefore, as the fats and oils produced by microalgae, fats and oils of which fatty acid species generated by hydrolysis can be utilized by a bacterium such as an L-amino acid-producing bacterium as a carbon source are preferred, and fats and oils highly containing such fatty acid species are more preferred. Examples of long chain fatty acid species utilizable by bacteria having an L-amino acid-producing ability include lauric acid, myristic acid, palmitic acid, stearic acid, oleic acid, and so forth.

Furthermore, besides fats and oils, organisms generally contain lipids, which release fatty acid(s) by hydrolysis. The fatty acid produced by hydrolysis of lipids can also be used as a carbon source. Examples of the lipids include waxes and ceramides, which are simple lipids; phospholipids and glycolipids, which are complex lipids; and so forth. Examples of the lipids also include terpenoids and steroids.

Fatty acids may be directly produced by a microalga. That is, a fatty acid, which is an example of objective substances, may be a fatty acid produced by hydrolysis of such a fat or oil, or lipid as mentioned above, or a fatty acid directly produced by a microalga.

The hydrolysate of a fat or oil refers to a hydrolysate obtained by hydrolyzing such a fat or oil of microalga as mentioned above by a chemical or enzymatic method. As the chemical hydrolysis method, the continuous high temperature hydrolysis method is generally performed, in which fats and oils and water are brought into contact with each other as countercurrents under a high temperature (250 to 260° C.) and a high pressure (5 to 6 MPa). Furthermore, it is known that hydrolysis of fats and oils occurs in the presence of a strong acid or an acid catalyst (U.S. Pat. No. 4,218,386). Furthermore, hydrolysis by a reaction at a low temperature (around 30° C.) using an enzyme is also industrially performed (Jaeger, K. E. et al., 1994, FEMS Microbiol. Rev., 15:29-63). As the enzyme, lipase, which is an enzyme catalyzing the hydrolysis reaction of fats and oils, can be used.

Specifically, for example, by putting equal amounts of fats and oils and water in a small pressure vessel, and stifling the mixture for about 1 hour with heating at 200° C., a hydrolysis rate of about 70 to 80% can be obtained. For the industrial reaction, high temperature (250 to 260° C.) and high pressure (5 to 6 MPa) conditions are used. On the other hand, according to the enzymatic method, the hydrolysis can be performed under milder conditions. A skilled artisan can easily perform such an enzymatic reaction with stirring water and fats and oils at a temperature suitable for the lipase reaction. Lipases are industrially important enzymes, and are industrially used in various ways (Hasan, F. et al., 2006, Enzyme and Microbiol. Technol., 39:235-251). One kind of enzyme or two or more kinds of enzymes may be used.

Lipases are enzymes that hydrolyze fats and oils into fatty acid(s) and glycerol, and are also called triacylglycerol lipases or triacylglyceride lipases.

Lipases have been found in various organisms. Lipases derived from any species may be used so long as they catalyze the aforementioned reaction. In recent years, various attempts have also been made to produce biodiesel fuels, which are fatty acid esters, from fats and oils and an alcohol by using a lipase enzyme (Fukuda, H., Kondo, A., and Noda, H., 2001, J. Biosci. Bioeng., 92, 405-416).

As typical lipases derived from microorganisms, many lipases derived from the genus Bacillus, Burkholderia, Pseudomonas, or Staphylococcus are known (Jaeger, K. E., and Eggert, T., 2002, Curt Opin. Biotechnol., 13:390-397).

The hydrolysate of a fat or oil is a mixture of fatty acid(s) and glycerol, and it is known that weight ratio of glycerol to fatty acid(s) contained in hydrolysates of a common fat or oil is about 10%. The hydrolysate may be a reaction product of the hydrolysis reaction as it is, or a fractionated or purified product of the reaction product, so long as it contains carbon sources utilizable by bacteria such as fatty acids and glycerol derived from fats and oils. When the hydrolysate contains fatty acid(s) and glycerol, the weight ratio of glycerol and fatty acid(s) (weight of glycerol:weight of fatty acid(s)) is 2:100 to 50:100, or 5:100 to 20:100.

The hydrolysate of a fat or oil usually separates into a lower layer containing glycerol (aqueous phase) and an upper layer containing fatty acid(s) (oil phase) at a temperature around room temperature. If the lower layer is collected, a fraction mainly containing glycerol is obtained. If the upper layer is collected, a fraction mainly containing fatty acid(s) is obtained. A hydrolysate of a fat or oil can be used as, for example, a carbon source for culturing a bacterium such as an L-amino acid-producing bacterium. Although any of glycerol and fatty acid(s) may be used as a carbon source, it is preferable to use both glycerol and fatty acid(s). When a hydrolysate of a fat or oil containing both glycerol and fatty acid(s) is used, it is preferable to subject the hydrolysate of a fat or oil to an emulsification treatment. Examples of the emulsification treatment include addition of an emulsification enhancer, stifling, homogenization, ultrasonication, and so forth. It is considered that the emulsification treatment makes it easier for bacteria such as L-amino acid-producing bacteria to utilize glycerol and fatty acid(s), and L-amino acid fermentation proceeds more effectively. The emulsification treatment may be any treatment that makes it easier for bacteria such as bacteria having an L-amino acid-producing ability to utilize a mixture of fatty acid(s) and glycerol. As the emulsification method, for example, addition of an emulsification enhancer or a surfactant can be contemplated. Examples of the emulsification enhancer include phospholipids and sterols. Examples of the surfactant include, as nonionic surfactants, polyoxyethylene sorbitan fatty acid esters such as poly(oxyethylene) sorbitan monooleic acid ester (Tween 80), alkyl glucosides such as n-octyl β-D-glucoside, sucrose fatty acid esters such as sucrose stearate, polyglyceryl fatty acid esters such as polyglycerin stearic acid ester, and so forth. Examples of the surfactant include, as ampholytic surfactants, N,N-dimethyl-N-dodecylglycine betaine, which is an alkylbetaine, and so forth. Besides these, surfactants generally used in the field of biology such as Triton X-100, polyoxyethylene(20) cetyl ether (Brij-58), and nonylphenol ethoxlate (Tergitol NP-40) can be used.

Furthermore, an operation for promoting emulsification and/or homogenization of hardly soluble substances such as fatty acids is also effective. This operation may be any operation that promotes emulsification and/or homogenization of a mixture of fatty acid(s) and glycerol. Specific examples thereof include stifling, homogenizer treatment, homomixer treatment, ultrasonic treatment, high pressure treatment, high temperature treatment, and so forth, and stifling, homogenizer treatment, ultrasonic treatment, and a combination of these are more preferred.

It is particularly preferable to perform the treatment with the aforementioned emulsification enhancer in combination with stirring, homogenizer treatment, and/or ultrasonic treatment. These treatments are desirably carried out under alkaline conditions, under which fatty acids are more stable. As the alkaline condition, pH of 9 or higher is preferred, and pH of 10 or higher is more preferred.

Production of the objective substance can be confirmed by known methods used for detection or identification of substances. Examples of such methods include, for example, HPLC, LC/MS, GC/MS, NMR, and so forth. For example, the concentration of glycerol can be measured with a kit such as F-Kit Glycerol (Roche Diagnostics), or any of various biosensors. Further, for example, the concentration of a fatty acid or fat or oil can be measured by gas chromatography (Hashimoto, K. et al., 1996, Biosci. Biotechnol. Biochem., 70:22-30) or HPLC (Lin, J. T. et al., 1998, J. Chromatogr. A., 808:43-49).

The culture method as described herein may include the step of collecting the objective substance. The objective substance can be collected by a method appropriately chosen according to various conditions such as the type of the objective substance. For example, an L-amino acid can be usually collected from the fermentation broth by a combination of conventionally known methods such as ion-exchange resin method (Nagai, H. et al., Separation Science and Technology, 39(16), 3691-3710), precipitation, membrane separation (Japanese Patent Laid-open (Kokai) Nos. 9-164323 and 9-173792), crystallization (WO2008/078448, WO2008/078646), and so forth. When an L-amino acid accumulates in cells, for example, the cells can be disrupted with ultrasonic waves or the like, and then the L-amino acid can be collected by the ion exchange resin method or the like from the supernatant obtained by removing the cells from the cell-disrupted suspension by centrifugation. Other substances can also be collected by the same methods as those for collecting an L-amino acid mentioned above.

### EXAMPLES

Hereafter, the present invention will be explained more specifically with reference to the following non-limiting examples.

### Example 1

### Culture of Microalga, Chlorella kessleri 11h Strain

The Chlorella kessleri 11h strain (UTEX 263) was obtained from the University of Texas at Austin, The Culture Collection of Algae (UTEX) (1 University Station A6700, Austin, Tex. 78712-0183, USA). The Chlorella kessleri 11h strain was cultured at a culture temperature of 30° C. and a light intensity of 7,000 luxes for 7 days with shaking in 100 mL of the 0.2×Gamborg's B5 medium (NIHON PHARMACEUTICAL) contained in a 500 mL-volume conical flask under a mixed-gas atmosphere of air and CO₂ containing 3% CO₂ in an incubator (CLE-303, culture apparatus produced by TOMY), and the resultant culture broth was used as a preculture broth. The preculture broth in a volume of 80 mL was added to 1.5 L of the 0.2×Gamborg's B5 medium contained in a 2 L-volume jar fermenter (Ishikawa Seisakusho), and culture was performed at a culture temperature of 30° C. and a light intensity of 20,000 luxes for 14 days with blowing 500 mL/minute of a mixed gas of air and CO₂ containing 3% CO₂ into the medium. As the light source, white light from a fluorescent lamp was used. The alga bodies obtained above are henceforth referred to as “alga biomass”.

### 0.2×Gamborg's B5 Medium:

| | | |
|---|---|---|
| KNO₃ | 500 | mg/L |
| MgSO₄•7H₂O | 50 | mg/L |
| NaH₂PO₄•H₂O | 30 | mg/L |
| CaCl₂•2H₂O | 30 | mg/L |
| (NH₄)₂SO₄ | 26.8 | mg/L |
| Na₂-EDTA | 7.46 | mg/L |
| FeSO₄•7H₂O | 5.56 | mg/L |
| MnSO₄•H₂O | 2 | mg/L |
| H₃BO₃ | 0.6 | mg/L |
| ZnSO₄•7H₂O | 0.4 | mg/L |
| KI | 0.15 | mg/L |
| Na₂MoO₂•2H₂O | 0.05 | mg/L |
| CuSO₄•5H₂O | 0.005 | mg/L |
| CoCl₂•6H₂O | 0.005 | mg/L |

The medium was sterilized by autoclaving at 120° C. for 15 minutes.

### Example 2

### Preparation of Residual Alga Bodies Obtained after Extraction of Alga-Derived Fats and Oils

Culture broth in a volume of 1.5 L of the Chlorella kessleri 11h strain cultured in the same manner as that of Example 1 was centrifuged at 5,000 rpm for 10 minutes, and then about 1.47 L of the supernatant was removed to prepare about 30 mL of an alga body concentrate. The alga bodies were suspended, the suspension was adjusted to pH 4.6 by adding 1 N hydrochloric acid, and the total volume of the suspension was made to be 40 mL by adding the centrifugation supernatant. This suspension was incubated at 50° C. for 6 hours with stirring. The reaction mixture after the incubation was centrifuged at 5,000 rpm for 10 minutes, and the supernatant was removed. The obtained precipitate was suspended in 39 ml of ethanol, and the suspension was incubated at 50° C. for 1 hour with stifling. The reaction mixture after the incubation was filtered to obtain a filtrate containing alga-derived fats and oils extracted from the alga bodies, and residual alga bodies as a residue remained on the filter paper. The filter paper and the residue on the filter paper were washed with 8 mL of ethanol, and the ethanol used for the washing was combined with the filtrate obtained above.

### Example 3

### Acid Hydrolysis of Alga Biomass and Residual Alga Bodies

The alga biomass obtained in Example 1 and the residual alga bodies obtained in Example 2 were each hydrolyzed by using sulfuric acid as follows.

The weight of the total solid content contained in the culture broth was measured for each of the culture broth obtained in Example 1, and the culture broth used for the preparation of the residual alga bodies in Example 2 according to the following procedures. A glass fiber filter paper was dried, and the weight thereof was measured. From the culture broth uniformized by sufficient stirring, a portion of 3 mL was accurately separated, and filtered through the aforementioned glass fiber filter paper. The glass fiber filter paper after the filtration was dried again, and the weight thereof was measured. The weight difference between the weights measured before and after the filtration was considered to be the weight of the total solid content contained in 3 mL of the culture broth. As a result, the weight of the solid content contained in the culture broth obtained in Example 1 was found to be 3.62 g/L. Further, the weight of the solid content contained in the culture broth used for the preparation of the residual alga bodies in Example 2 was 3.79 g/L.

In order to prepare an acid hydrolysis product of the alga biomass, 1.3 L of the culture broth obtained in Example 1 was centrifuged at 5,000 rpm for 10 minutes, and about 1.27 L of the supernatant was removed to obtain about 30 mL of an alga biomass concentrate (alga body concentrate). The solid content concentration thereof was calculated to be about 159 g/L on the basis of the solid content concentration and concentration rate of the culture broth.

Further, in order to prepare acid hydrolysis product of the residual alga bodies, the residue after the lipid extraction obtained in Example 2 (residual alga bodies) was suspended in reverse osmotic water to obtain about 40 mL of a residual alga body concentrate. The solid content concentration thereof was similarly calculated to be about 125 g/L.

On the basis of the nitrogen amount contained in each concentrate, sulfuric acid was added thereto so that the molar ratio of sulfate ions to nitrogen (SO₄/N) became 10 after the addition. At this point, the solid content in the reaction mixture for the residual alga bodies was about 11%, and the solid content in the reaction mixture for the alga biomass (alga bodies) was about 13%.

Each reaction mixture was treated at 116° C. for 32 hours. The resultant was adjusted to pH 6.0 by adding 12 N KOH, and the insoluble matter was removed by filtration. The obtained filtrates are referred to as “acid hydrolysis product of alga body biomass”, and “acid hydrolysis product of residual alga bodies”, respectively.

### Example 4

### Culture of Microalga, Chlorella kessleri 11h Strain, Using Acid Hydrolysis Product of Alga Biomass and Acid Hydrolysis Product of Residual Alga Bodies

Each of the acid hydrolysis product of alga biomass and the acid hydrolysis product of residual alga bodies obtained in Example 3 was added to a medium to be used for culture of an alga as follows.

The Chlorella kessleri 11h strain was cultured at a culture temperature of 30° C. and a light intensity of 7,000 luxes for 7 days with shaking in 10 mL of the 0.2×Gamborg's B5 medium (NIHON PHARMACEUTICAL) contained in a 50 mL-volume conical flask under a mixed-gas atmosphere of air and CO₂ containing 3% CO₂ in an incubator (CLE-303, culture apparatus produced by TOMY), and the resultant culture broth was used as a preculture broth. The preculture broth in a volume of 0.5 mL was added to 10 mL of the 0.2×Gamborg's B5 medium added with 0.1 mM (in terms of nitrogen concentration) of the acid hydrolysis product of alga biomass or the acid hydrolysis product of residual alga bodies, which was contained in a 50 mL-volume conical flask, and culture was performed at a culture temperature of 30° C. and a light intensity of 7,000 luxes for 5 days with shaking under a mixed-gas atmosphere of air and CO₂ containing 3% CO₂ in an incubator (CL-301, culture apparatus produced by TOMY).

For comparison of the growth, the culture broth in a volume of 30 μL was taken from the flask as a sample during the shaking culture in a sterile manner, the sample was diluted 10 times with reverse osmotic water, and the turbidity (750 nm) of the diluted sample was measured.

The amount of the accumulated fatty acids was measured as follows. The culture broth in a volume of 500 μL was taken from the flask as a sample during the shaking culture in a sterile manner, frozen at −80° C. for 30 minutes, and then immediately treated at 50° C. for 20 hours. Then, the sample was centrifuged at 12,000 rpm at 4° C. for 5 minutes, and the supernatant was removed to obtain precipitate of alga bodies. A mixed solution of methanol and chloroform (1:1) in a volume of 500 μL was added to the precipitate, and the mixture was shaken for 20 minutes to extract lipids. The mixed solution of methanol and chloroform containing the extracted lipids was concentrated by centrifugation, and the fatty acid concentration in a sample obtained by dissolving again the concentrate in isopropanol was measured by using a fatty acid quantification kit (LabAssay NEFA, Wako Pure Chemical Industries).

The results are shown in Tables 1 and 2. As shown in Tables 1 and 2, when the medium added with the acid hydrolysis product of alga biomass or the acid hydrolysis product of residual alga bodies was used, better growth and a higher fatty acid accumulation amount were obtained as compared with the case of using the medium not added with either the acid hydrolysis product of alga biomass or the acid hydrolysis product of residual alga bodies.

Table 1

TABLE 1
OD₇₅₀ (×10) on day 5 of culture

| | | Sulfuric acid |
|---|---|---|
| Addition | Sulfuric acid hydrolysis product | hydrolysis product |
| amount | of alga biomass | of residual alga bodies |
| 0 mM | 0.641 | 0.641 |
| 0.1 mM | 0.652 | 0.667 |

Table 2

TABLE 2
Fatty acid accumulation (mg/L) on day 5 of culture

| | | Sulfuric acid |
|---|---|---|
| Addition | Sulfuric acid hydrolysis product | hydrolysis product |
| amount | of alga biomass | of residual alga bodies |
| 0 mM | 112 | 112 |
| 0.1 mM | 155 | 146 |

### INDUSTRIAL APPLICABILITY

According to the present invention, a nutrient additive effective for culturing a microorganism or alga can be produced. By using the nutrient additive produced according to the present invention, a microorganism or alga can be cultured at a lower cost. Further, in an embodiment, by using the nutrient additive produced according to the present invention, a microorganism or alga can be cultured at a lower cost thereby to produce a desired objective substance.

While the invention has been described in detail with reference to preferred embodiments thereof, it will be apparent to one skilled in the art that various changes can be made, and equivalents employed, without departing from the scope of the invention. Each of the aforementioned documents is incorporated by reference herein in its entirety.

1. A method for producing a nutrient additive useful in culturing a microorganism or a microalga, comprising:
a) culturing a microalga in a medium to produce biomass;
b) hydrolyzing the biomass with an acid, producing an hydrolysate; and
c) preparing a nutrient additive comprising the hydrolysate,
wherein the acid is selected from the group consisting of sulfuric acid, hydrochloric acid, nitric acid, and phosphoric acid.

2. The method according to claim 1, wherein the hydrolysate promotes growth of a microorganism or microalga.

3. The method according to claim 1, wherein the acid is sulfuric acid, and the acid is added in such an amount that the molar ratio of sulfate ions to nitrogen contained in the biomass (SO₄/N) is 0.1 to 10.

4. The method according to claim 1, wherein the acid is hydrochloric acid, and the acid is added in such an amount that the molar ratio of chloride ions to nitrogen contained in the biomass (Cl/N) is 0.1 to 20.

5. The method according to claim 1, wherein the acid is phosphoric acid, and the acid is added in such an amount that the molar ratio of phosphate ions to nitrogen contained in the biomass (PO₄/N) is 0.1 to 100.

6. The method according to claim 1, wherein the acid is nitric acid, and the acid is added in such an amount that the molar ratio of nitrate ions to nitrogen contained in the biomass (NO₃/N) is 0.1 to 100.

7. The method according to claim 3, wherein the acid is sulfuric acid, and the acid is added in such an amount that the molar ratio of sulfate ions to nitrogen contained in the biomass (SO₄/N) is 0.8 to 3.

8. The method according to claim 1, wherein said hydrolyzing is performed at 75 to 130° C. for 5 to 50 hours.

9. The method according to claim 1, wherein said hydrolyzing is performed at 110 to 120° C. for 10 to 32 hours.

10. The method according to claim 1, comprising treating the biomass at 80 to 110° C. for 30 minutes to 2 hours before said hydrolyzing.

11. The method according to claim 1, comprising treating the biomass at 90 to 105° C. for 40 to 90 minutes before said hydrolyzing.

12. A method for producing objective substance(s), comprising:
culturing a microalga or a microorganism in a medium comprising the nutrient additive produced by the method according to claim 1.

13. The method according to claim 12, wherein the objective substance(s) is L-amino acid(s).

14. The method according to claim 12, wherein the objective substance(s) is starch.

15. The method according to claim 12, wherein the objective substance(s) is lipid(s) or fatty acid(s).
